(19) United States
(12) Patent Application Publication
(10) Pub. No.: US 2004/0096974 A1
(43) Pub. Date: May 20, 2004

(54) Methods and materials for generating genetic disruptions in bacterial cells

(76) Inventors: Paul Herron, Swansea (GB); Paul J. Dyson, Swansea (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/632,398

(22) Filed: Jul. 31, 2003

Related U.S. Application Data
(60) Provisional application No. 60/399,751, filed on Jul. 31, 2002.

(51) Int. Cl. C12N 15/74

(52) U.S. Cl. 435/473

(57) ABSTRACT

The present invention provides materials and methods for introducing genetic disruptions in bacterial genetic material, especially in that of Streptomyces spp.. A novel transposon is provided, which has an origin of transfer between inverted repeat sequences. The transposon may also include a genetic marker. The transposon introduces a disruption into DNA of interest, which disruption may then be conjugated into host bacteria, including bacteria of other species or strains. The host bacteria is incubated at conditions suitable for homologous recombination between the conjugated DNA and the host DNA. The effect of the disruption in different genetic backgrounds can therefore be investigated. The disruption may be stored as a mobile genetic element ready for transfer to a test host.

The present application claims priority to U.S. Provisional Application 60/399,751 filed Jul. 31, 2002, the entire disclosure of which is incorporated by reference herein.

### FIELD OF THE INVENTION

The present invention relates to methods and materials for generating genetic disruptions in bacterial genetic material, especially genetic disruptions in genetic material of Streptomyces spp..

The most preferred genetic material for study is that of S. coelicolor M145, a plasmid-free (SCP1⁻SCP2⁻) derivative of the wild type S. coelicolor A3(2) strain. Streptomyces coelicolor A3(2) is genetically the most studied Streptomyces species. It is for this reason that the entire single, linear, 8,800 kb chromosome of S. coelicolor A3(2) was sequenced (using S. coelicolor M145) at the Sanger Centre. 7825 genes were predicted, with an average gene size of 1.1 kb. 53% of these 7825 genes have no known function. Most of these genes are probably non-essential for growth under normal laboratory conditions. It is of great interest to study these genes, and to this end it is of interest to generate mutants containing disruptions in different non-essential genes (and/or their control sequences), resulting in mutations (often knock-out mutations) of those genes.

Once an interesting mutation has been identified, it may also be of interest to introduce it into other species or strains, to investigate its effect in different genetic backgrounds.

It may also be of interest to study the control of genes containing interesting mutations.

In the work leading to the present invention, the inventors have constructed a novel transposon, designated Tn5062, which contains an origin of transfer, a three frame translational stop sequence, an antibiotic resistance marker and a promoterless copy of the enhanced green fluorescent protein gene (EGFP) between Tn5-like inverted repeat sequences.

Transposition of this novel transposon was performed in vitro into cosmid DNA from an S. coelicolor cosmid library, using the in vitro transposition protocol of Epicentre (Madison, Wis., USA), and the transposed cosmid DNA was used to transform E. coli cells. Transposition target sites from different transposition events were determined by sequencing using transposon-specific primers and transposed cosmid DNA as the template.

Replacement of a wild-type gene with a disrupted, cosmid-borne copy was effected by conjugation from E. coli, followed by homologous recombination, and determined by marker replacement.

The methods and materials of the invention and various preferred embodiments offer certain benefits, which are not provided by other previously known mutagenesis techniques that use transposons (such as that disclosed in PCT/GB02/02884). In particular, the inclusion of a step of conjugating transposed DNA allows the same mutation to be transferred into multiple genetic backgrounds (e.g. different actinomycete species or strains); it also allows more convenient identification of the site of transposition than if transposition were carried out in the host cell; it also allows more flexibility in storage of the mutation, e.g. as purified cosmid DNA or in E. coli cells; it is also advantageous in the absence of a reliable generalised transduction system (as is the case for Streptomyces, although electroporation has been demonstrated for some species). The invention is preferably applied to the mutagenesis of DNA from a species whose genome has been sequenced (such as S. coelicolor) or a related species.

Accordingly, in a first aspect, the invention provides a nucleic acid construct comprising inverted repeat sequences of a transposable element and an origin of transfer, wherein the origin of transfer lies between the inverted repeat sequences, such that a transposition event involving the inverted repeat sequences will result in the origin of transfer being included in the resultant insertion at the transposition target site.

In a second aspect, the invention provides a method for mutagenising DNA of interest from a bacterial species, the method comprising:

(a) contacting said DNA of interest with a nucleic acid construct of the invention, to form a transposition mixture;

(b) incubating the transposition mixture under conditions suitable for transposition to occur, said contacting and incubating steps being performed other than within cells of said bacterial species;

(c) transferring transposed DNA of said transposition mixture by conjugation from a donor bacterial cell into a host bacterial cell; and

(d) incubating the host cell under conditions suitable for homologous recombination between the transposed DNA and the DNA of the host cell.

In practice, the method will generally involve the use of multiple donor and host cells.

For the avoidance of doubt, it is hereby stated that the term “inverted repeat sequences” refers to the short (typically about 10-40 bp) terminal inverted repeat sequences of an insertion sequence (IS element) or class II transposon, which interact with a transposase to mediate transposition. It does not refer to an entire IS element from a class I transposon. Class I transposons consist of one or more structural genes flanked on either side by an IS element (which may be identical or different). For example, Tn5 consists of two IS elements (designated “IS50L” and “IS50R”) flanking various structural genes. Each IS element of a class II transposon therefore includes inverted repeat sequences.

Especially preferred inverted repeat sequences are the 19 bp “Mosaic Ends” of the EZ::TN™ system of Epicentre, as used in the Example and labelled “OE-L” and “OE-R” in FIG. 1, having the sequence:

5′-CTGTC TCTTA TACAC ATCT-3′

3′-GACAG AGAAT ATGTG TAGA-5′

These specific inverted repeat sequences are recognised by the well-known and well-characterised hyperactive mutant Tn5 transposase for high frequency transposition. This mutant transposase is commercially available (e.g. from Epicentre, as EZ::TN™ transposase)

However, it is contemplated that many other inverted repeat sequences may be used in the practice of the invention. The skilled person will be aware of other transposons, the inverted repeat sequences of which may also be used, in conjunction with a transposase enzyme capable of recognising the inverted repeat sequences (see e.g. Singleton and Sainsbury (1987) Dictionary of Microbiology and Molecular Biology, 2nd edition, John Wiley & Sons, under the entry “transposable element” and other entries referred to therein, as well as references cited in those entries, e.g. the review Grindley and Read (1985) and Shapiro (ed) (1983) “Mobile Genetic Elements”, Academic Press; see also Berg and Howe (1989) and Kieser et al (2000)). Indeed several transposon systems are commercially available, each comprising a transposon and a transposase capable of recognising and mediating transposition of the transposon. These and other publicly known transposons could readily be adapted for use in accordance with the invention. Examples of commercially available systems include the Genejumper™ system of Invitrogen Corporation (Carlsbad, Calif., USA), based on the bacteriophage μA transposon; the μA transposon system of Finnzymes Oy (Espoo, Finland); and the GPS system of New England Biolabs (Beverly, Mass., USA), based on the Tn7 transposon.

Generally, the inverted repeat sequences and transposase will originate from the same transposon or related transposons. Also contemplated and within the scope of the invention are variant inverted repeats and/or transposases, which remain capable of interacting with each other to mediate transposition.

Generally preferred inverted repeat sequences are, or are derived from, the OE and/or IE inverted repeat sequences of the transposon Tn5 (from which the EZ::TN™ Mosaic Ends are themselves derived). The OE sequence is 5′-CTGAC TCTTA TACAC AAGT; the IE sequence is 5′-CTGTC TCTTG ATCAG ATCTT GATC. Tn5 has the most random insertion pattern of known transposons. This property is shared by the EZ::TN™ Mosaic Ends. Such inverted repeats will generally be used with native Tn5 transposase (though this is not suitable for in vitro transposition) or, preferably, the commercially available hyperactive mutant Tn5 transposase (e.g. of Epicentre), which is suitable for in vitro use. The mutant Tn5 transposase is capable of recognising both the Mosaic Ends and the wild-type Tn5 inverted repeat sequences.

Tn5-like inverted repeat sequences preferably display at least 70%, 75%, 80%, 85%, 90% or 95% sequence identity with any one or more of the Tn5 OE sequence, the Tn5 IE sequence and the Mosaic Ends, a comparable level of identity to that shown between the OE sequence and the Mosaic Ends (16 identical bases out of 19) and between the IE sequence and the Mosaic Ends (15 identical bases out of 19 in the shorter sequence). Percentage sequence identity is defined as the percentage of nucleic acid residues in the shorter of the sequences under comparison that are identical to corresponding nucleic acid residues in the other sequence, when the sequences are aligned. Up to a total of 5 gaps may be included in one or both sequences to optimise the alignment. Like the Mosaic Ends, a Tn5-like sequence may be a hybrid of the OE and IE sequences, each residue of the Tn5-like sequence being selected from the residues found at the corresponding position in either the IE or the OE sequences.

The inverted repeat sequences preferably do not demonstrate high target site specificity. This provides the advantage of allowing essentially untargeted gene disruption to occur, rather than bias towards disruption at a limited number of locations possessing the relevant specific target site. The Tn3 and IS1 transposable elements show preference towards AT-rich regions. Such target site specificity is not regarded to be high for the present purposes and transposable elements having Tn3-like or IS1-like inverted repeat sequences are within the scope of this preference. Both Tn3 and Tn10 transposable elements display hotspots for insertion, at which insertion occurs with greater frequency than at other locations, so some bias towards these hotspots might be expected. Hotspots for Tn3 have homology with the Tn3 inverted repeat sequences; those for Tn10 bear no obvious relationship to the Tn10 inverted repeat sequences. Such target site specificity is also not regarded to be high for the present purposes and transposable elements having Tn3-like or Tn10-like inverted repeat sequences are also within the scope of this preference. By contrast, the IS5 transposable element inserts only at sites containing the target site C(T/A)A(G/A). This level of specificity is not within the scope of this preference.

Particularly when transposition is intended to occur within a bacterial cell (see below), the construct may encode all the functionalities necessary for transposition to occur, e.g. a transposase (which will usually originate from the same transposable element as the inverted repeat sequences). Some transposable elements, which transpose by a replicative mechanism, also require a resolvase gene and an internal resolution site for transposition to occur. These may be included if required.

Preferably, however, the construct is intended for use in transposition in vitro, i.e. outside any bacterial cell. In such cases, it preferably does not encode a transposase. Rather, the transposase protein is added to the transposition reaction mixture. Again, the transposase will usually originate from the same transposable element as the inverted repeat sequences. Preferred transposase is the commercially available hyperactive mutant Tn5 transposase.

Many sequences for the Tn5 transposase are available, e.g. via GenBank. Goryshin and Rezinkoff (1998) describes a hyperactive form of the transposase, on which the EZ::TN™ transposase system is based. See also Davies et al. (2000), which reports the 3D structure of the enzyme-DNA complex.

Particularly when the construct is intended to be used for transposition in vitro, the inverted repeat sequences are preferably of, or derived from the inverted repeat sequences of, a transposable element that employs a non-replicative (e.g. a cut-and-paste) transposition mechanism. The Tn5 transposon replicates in this manner; a further example is Tn10.

A transposition event involving the construct of the invention will lead to the insertion of the origin of transfer into the transposition target site. If the transposition target site is in a circular DNA molecule, such as a cosmid or other plasmid, the circular DNA molecule can then be mobilised from one bacterial cell (the donor cell) into another bacterial cell (the host cell) by conjugation. The DNA of interest is therefore preferably contained in one or more circular DNA molecules (typically a large number of such molecules), such as a cosmid or other plasmid.

Preferably the origin of transfer is an oriT which can be mobilised by the helper plasmids pUZ8002 and pUB307, such as an oriT from an IncP-group plasmid, such as RP4 (also designated RP1/RK2; Pansegrau et al., 1994), preferably having the nucleic acid sequence:

CCGGGCAGGA TAGGTGAAGT AGGCCCACCC GCGAGCGGGT GTTCCTTCTT

CACTGTCCCT TATTCGCACC TGGCGGTGCT CAACGGGAAT CCTGCTCTGC

GAGGCTGGC,

or a variant thereof having origin of transfer function. However, the use of any other suitable origin of transfer is also contemplated.

Preferably the construct comprises a selectable marker gene (such as an antibiotic resistance gene), located between the inverted repeat sequences. The presence of a selectable marker in the insertion following a transposition event allows convenient identification of transposed DNA.

Preferably the construct comprises a reporter gene (preferably a promoterless reporter gene), located between the inverted repeat sequences. Insertion of a promoterless reporter gene into a target site downstream of a promoter will allow analysis of gene expression under the control of that promoter. Preferably the promoterless reporter gene is operatively associated with a ribosome binding site. Preferably the construct further comprises, upstream of the reporter gene and ribosome binding site and between the inverted repeat sequences, a translational stop sequence (preferably a three-frame translational stop sequence). Expression of a fusion protein (of the partial gene product of the mutagenised gene fused to the reporter gene product, or a nonsense product of the reporter gene) may interfere with expression of the reporter gene from its own ribosome binding site. This will be prevented by the presence of the translational stop sequence.

Preferred reporter genes are visible or visualisable. Especially preferred are genes for fluorescent proteins, e.g. those commercially available from BD Biosciences Clontech (Palo Alto, Calif., USA, a division of Becton Dickinson, Franklin Lakes, N.J., USA), such as enhanced green fluorescent protein (EGFP).

The construct may be linear, and may consist essentially of the inverted repeat sequences and any sequences located therebetween (i.e. the inverted repeat sequences may lie essentially at respective termini of the linear nucleic acid molecule), optionally with short sequences outside the inverted repeat sequences (e.g. sequences containing PCR primer binding sites and/or restriction sites, particularly sequences of 100 bp or less, 75 bp, 50 bp, 30 bp, 20 bp, 15 bp or 10 bp). The construct may in particular lack an origin of replication.

The construct may, however, be included within a vector, e.g. a plasmid. Typically, such a vector will include convenient PCR primer binding sites and/or restriction sites for the amplification or excision from the vector of a linear nucleic acid consisting essentially of the construct. In preferred embodiments of the invention, the construct will be intended for use, as such a linear molecule, in an in vitro transposition reaction. Apart from (and outside of) the construct of the invention, the vector may typically include an origin of replication and/or a selective marker.

The contacting and incubating steps (a) and (b) of the method of the invention may occur inside the donor (e.g. E. coli) cell, especially by transferring (e.g. by electroporation) the construct of the invention into cells containing a DNA of interest (e.g. E. coli cells containing an actinomycete cosmid). A preferred construct of the invention does not encode transposase, but may be pre-incubated with transposase to form a stable complex (referred to as a “transposome” in Epicentre literature), which may then be transferred into the donor cell (again, e.g. by electroporation) for the contacting step. Transposase enzymes typically require the presence of a metal ion (e.g. Mg²⁺ for Tn5 and the EZ::TN system) for transposition to occur. The pre-incubation step will therefore generally be performed in the absence of such an ion.

More preferably, however, the contacting and incubating steps (a) and (b) occur outside any bacterial cell, and the method comprises the further step (b1) of transferring the transposed DNA of the transposition mixture into the bacterial donor cell (preferably an E. coli cell), prior to the conjugation step (c) into the host cell. This has the advantage that transposition into the donor cell genome will be avoided. In this case also, the incubating step (b) will be carried in the presence of transposase (and any necessary metal ion, such as Mg²⁺ for Tn5 transposase, or the hyperactive mutant thereof). The step (b1) of transferring the transposed DNA of the transposition mixture into the donor cell may for example be accomplished by electroporation using the transposition mixture (optionally after stopping the transposition reaction, e.g. by denaturing the transposase). Following step (b1), the method preferably includes the step (b2) of detecting whether the donor cell has taken up transposed DNA. This may involve identifying in the donor cell the presence of a selectable marker gene included within the construct of the invention.

Particularly when the sequence of the DNA of interest is known (as it is for e.g. S. coelicolor A3(2)), the method may comprise an additional step of identifying the site in the DNA of interest at which a transposition event has led to an insertion. This may involve sequencing, preferably using a sequencing primer that binds to a site within the construct of the invention. The first sequence data will then correspond to a partial sequence of the construct, ending with one of the inverted repeat sequences. This will be followed by sequence data corresponding to the insertion site in the DNA of interest. Following a transposition event, the insertion site in the DNA of interest will be duplicated, the two copies of the insertion site being separated by the inverted repeat sequences and all sequences of the construct that lie between the inverted repeat sequences.

The insertion site can be identified after conjugation into the host cell and homologous recombination with the native gene. However, this requires isolation and manipulation of genomic host DNA, rather than the DNA of interest, which may be of smaller scale than the entire host genome. Accordingly, the insertion site is preferably identified before the conjugation step.

The construct may be designed to include one or more suitable sequencing primer binding sites, preferably located close to the inverted repeat sequences.

Preferably the DNA of interest is a DNA from a bacterial library. Preferably the bacterium from which the library is generated is an actinomycete, more preferably a streptomycete, more preferably a bacterium of the genus Streptomyces, more preferably of the species S. coelicolor, more preferably of the strain S. coelicolor A3(2).

For transfer of transposed DNA of interest bearing an RP4 oriT to occur by conjugation, a transfer function should be supplied, preferably in trans, e.g. by an E. coli donor strain such as ET12567 carrying the self-transmissible pUB307 (Bennett et al., 1977, Flett et al., 1997) or ET12567 carrying the non-transmissible pUZ8002 (Kieser et al., 2000).

Thus step (c) preferably includes such provision in trans of transfer function. This step may involve transforming the transposed DNA of interest (e.g. transposed cosmid) into a donor strain carrying a non-transmissible transfer plasmid (e.g. ET12567/pUZ8002), followed by incubation under suitable conditions with the host cell.

The host cell is preferably an actinomycete cell, more preferably a streptomycete cell, more preferably a cell of the genus Streptomyces, more preferably a cell of the species S. coelicolor, more preferably a cell of the strain S. coelicolor A3(2). The host cell is preferably a pre-germinated spore.

The host cell will frequently be of the species or strain from which the DNA of interest originates (particularly S. coelicolor). It is a particular advantage of the invention, however, that the same mutation can be introduced by conjugation into different host cells, which may be of different species or strains, such as different streptomycete strains.

For certain embodiments, which use an actinomycete bacterial host cell, the present invention provides the advantages that introduction of the mutagenised genetic material can be accomplished without protoplast transformation and regeneration, using conjugation into pre-germinated spores, and that (especially with conjugation from E. coli) the procedure is broadly applicable in introducing DNA into actinomycetes other than S. coelicolor (Matsushima et al., 1994). As well as being laborious, protoplast transformation and regeneration procedures may produce mutations.

The donor cell is preferably of a different cell type (i.e. of a different bacterial class, or higher taxonomical ranking) from the host cell; any convenient bacterial cell may be used. For convenience, however, the donor cell is most preferably an E. coli cell.

Where the host cell has methylation-specific restriction system (e.g. S. coelicolor has such a system, although the related strain S. lividans does not, MacNeil et al., 1992), the donor cell is preferably methylation-deficient, e.g. E. coli strain ET12567 (MacNeil et al., 1992).

The method may comprise an additional step (e) of detecting whether homologous recombination has occurred in the host cell. This may for example be indicated by the loss in the host cell of a selectable marker that is borne by the DNA (e.g. cosmid) of interest, but the retention of a selectable marker that is borne by the construct of the invention. This may for example be determined by replica plating.

The method may comprise an additional step, prior to the conjugation step, of replacing part or all of the transposition-derived insert by a further step of homologous recombination, to remove sequences from the insert and/or to add sequences to the insert, e.g. to include different marker genes in the insertion and/or to generate an in-frame translational fusion of an interrupted host cell coding sequence and a reporter gene in the insertion (e.g. by removing the translational stop sequence and reporter gene ribosome binding sequence). This step is preferably performed in a cell (preferably an E. coli cell) induced for Red-mediated recombination, e.g. on a transposed cosmid that has been transformed into the cell.

The mutant host cells produced according to this method may be stored for future use, in any suitable form, e.g. (when the host cell is an actinomycete) as spores. However, it may be more convenient to store mutagenised DNA of interest in the donor cells, other transformed bacterial cells not necessarily suitable for use as a conjugation donor (e.g. E. coli cells) or simply as DNA, e.g. in the form of isolated cosmids.

Accordingly, in a third aspect, the invention provides a method for mutagenising DNA of interest of a bacterial species, the method comprising the steps of:

contacting said DNA of interest with a nucleic acid construct of the invention, to form a transposition mixture;

incubating the transposition mixture under conditions suitable for transposition to occur, said contacting and incubating steps being performed other than within cells of said bacterial species; and

storing transposed DNA of said transposition mixture for future use in a method comprising transferring said transposed DNA from a donor bacterial cell into a host bacterial cell by conjugation and incubating the host cell under conditions suitable for homologous recombination between the transposed DNA and the DNA of the host cell.

In a fourth aspect, the invention provides a method for mutagenising DNA of interest of a bacterial species, the method comprising, following the production and storage of transposed DNA according to the third aspect of the invention, the steps of:

(c) transferring said transposed DNA by conjugation from a donor bacterial cell into a host bacterial cell; and

(d) incubating the host cell under conditions suitable for homologous recombination between the transposed DNA and the DNA of the host cell.

The present invention also provides a host cell producible or as produced by the process of the second and/or fourth aspect. Furthermore, the invention provides a method of determining the effect of a genetic disruption, the method comprising culturing such a host cell and determining the effect of the disruption on the cell.

The present invention also provides transposed DNA of interest producible or as produced by the process of the third aspect, optionally contained in a bacterial cell or cells (e.g. E. coli).

Preferably the method of the second aspect will be carried out simultaneously on several DNA molecules of interest (e.g. copies of a cosmid), which are conjugated from different donor cells into different host cells, to produce a plurality of independently mutated host cells.

Except where the context requires otherwise, all preferred features referred to herein are applicable to all aspects of the invention.

### BRIEF DESCRIPTION OF THE DRAWINGS

Embodiments of the invention will now be described, by way of example only, with reference to the accompanying drawings, in which:

FIGS. 1A and 1B show the sequence of transposon Tn5062, a nucleic acid construct according to the first aspect of the invention, along with the location of various components;

FIG. 2 shows the construction strategy for Tn5062.

### DETAILED DESCRIPTION OF THE INVENTION

### EXAMPLE

Summary

A procedure for efficient systematic mutagenesis of streptomycete genes is described. The technique employs in vitro transposon mutagenesis, using a novel transposon Tn5062. Mutations are initially derived in cloned streptomycete DNA propagated in Escherichia coli. The mutations are then mobilised into a streptomycete host in which marker replacement by homologous recombination occurs. The incorporation of a promoter-less copy of the eGFP reporter gene in Tn5062 permits temporal and spatial analysis of expression of a transposon tagged gene.

Bacterial strains, plasmids and oligonucleotides

Streptomyces coelicolor M145 was used as a test strain for transposition and was cultured on SFM agar using standard techniques according to Kieser et al. (2000). All DNA manipulations were carried out using Escherichia coli JM109 (Yanisch-Perron et al., 1985) or Escherichia coli Sure (Stratagene). E. coli was grown on either L agar plates, L broth or 2xYT broth (Sambrook et al. 1989). E. coli ET12567 (MacNeil et al., 1992) carrying pUZ8002 (Kieser et al., 2000) was used as a host to mobilize transposed cosmids into S. coelicolor M145. Oligonucleotides and plasmids/cosmids used in this study are listed in tables 1 and 2 respectively.

DNA manipulations

Plasmid isolations were carried out using Wizard SV kits from Promega and cloning steps were performed using established procedures. Restriction endonucleases and T4 ligase (obtained from New England Biolabs, Life Technologies or Promega) were used according to the manufacturer's instructions.

Construction of Tn5062

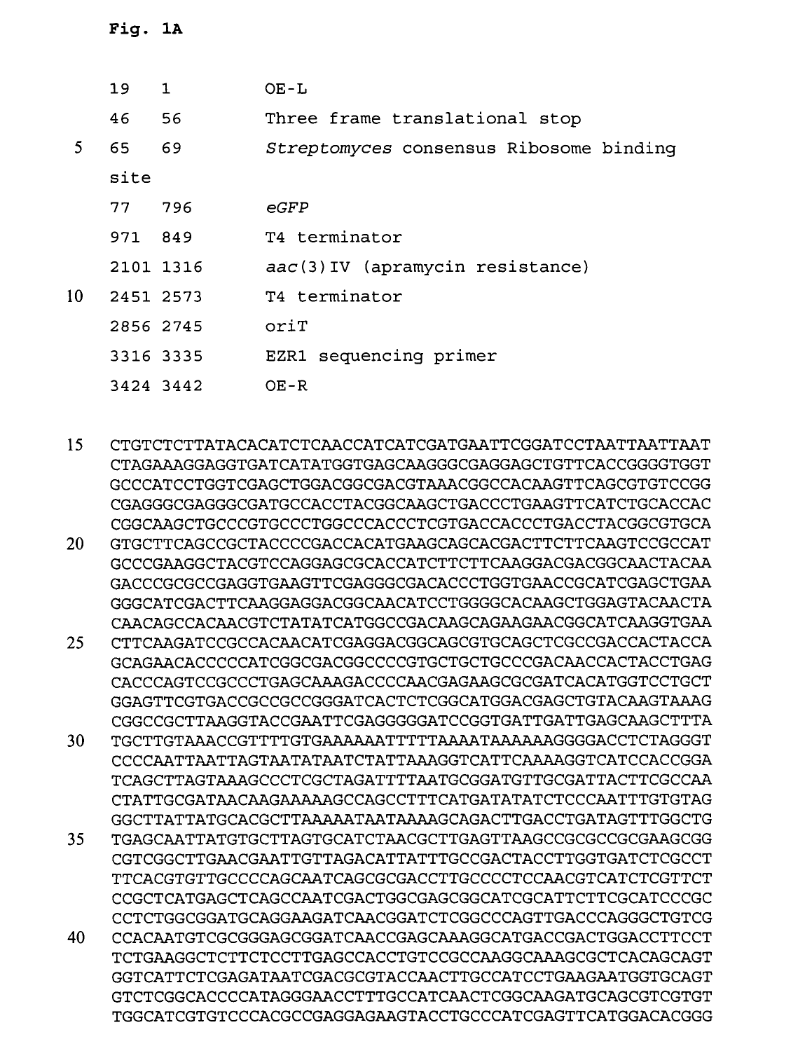

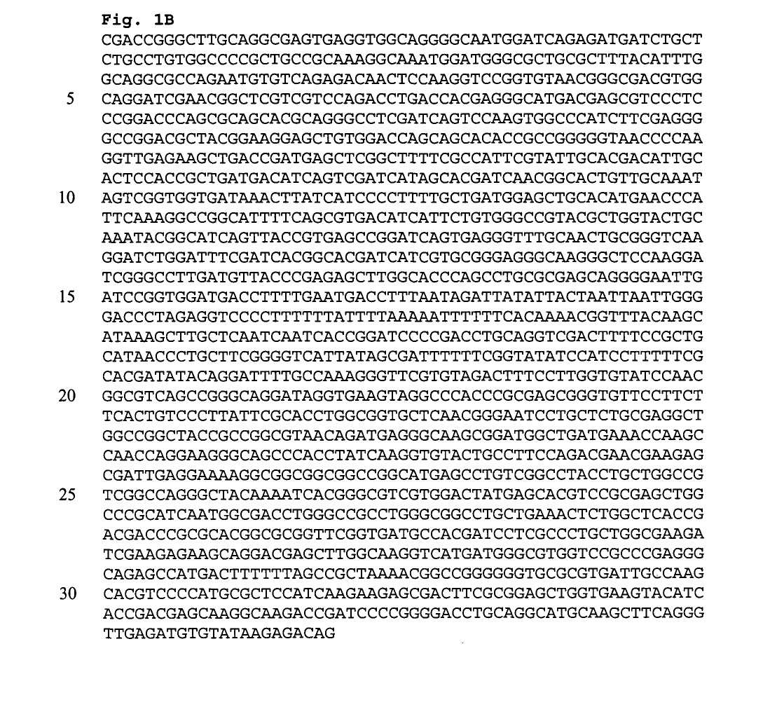

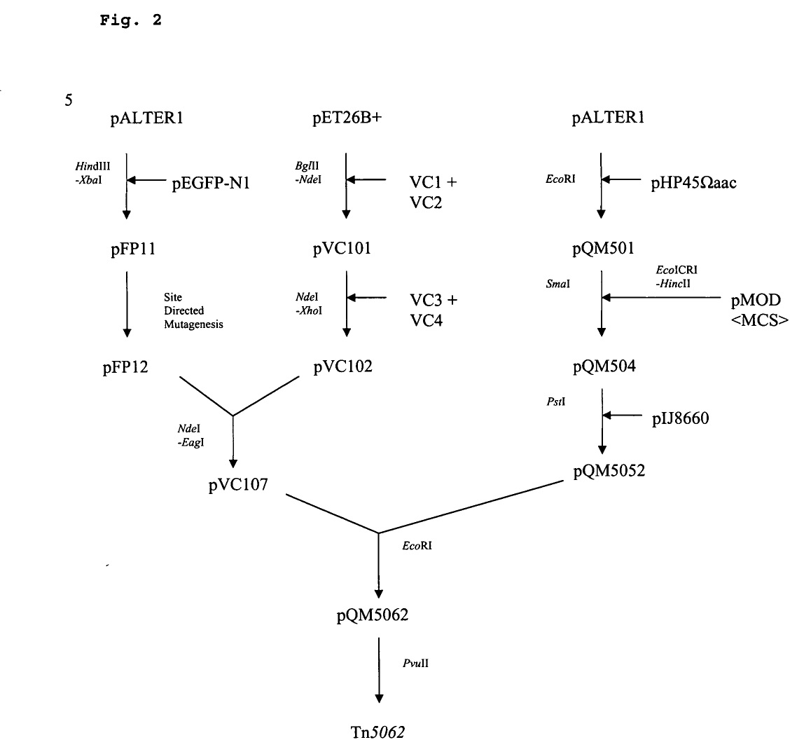

The first step in the construction of the transposon, Tn5062 (FIG. 1), was to clone eGFP from pEGFP-N1 to pALTER1 as a 787 bp HindIII-XbaI fragment creating pFP11 (FIG. 2). This allowed an NdeI site to be introduced at the start codon of eGFP by site directed mutagenesis using the altered sites kit from Promega according to the manufacturer's instructions resulting in pFP12 (ACCATG (pFP11) was changed to CATATG (pFP12); where ATG is the first codon of the eGFP gene). The three frame translational stop was constructed as a linker made from the oligonucleotides VC1 and VC2 (MWG-Biotech) and cloned into BglII-NdeI digested pET26B+ creating pVC101. This was digested with NdeI and XhoI and ligated to a second linker synthesised as the oligonucleotides VC3 and VC4 carrying a Streptomyces consensus ribosome binding site creating pVC102. eGFP was cloned from pFP12 into pVC102 as 725 bp NdeI-EagI fragment giving pVC107. aac3(IV) was first moved to pALTER1 from pHP45Ωaac as a 1783 bp EcoRI fragment creating pQM501. This plasmid was digested with SmaI and the 1794 bp aac3(IV) fragment introduced between the Tn5 inverted repeats of pMOD<MCS> by blunt-ended ligation with EcoICRI and HincII digested pMOD<MCS> resulting in pQM504. oriT was introduced into pQM504 as a 786 bp pstI fragment from pIJ8660 giving pQM5052. Finally eGFP was added to pQM5052 as a 782 bp EcoRI fragment from pVC107. This plasmid was named pQM5062 and allows Tn5062 to be liberated from the plasmid backbone by digestion with PvuII as a 3442 bp fragment (FIG. 1). The sequence of the transposon was verified by restriction digestion with appropriate enzymes and sequencing using a Beckman-Coulter CEQ 2000XL sequencer according to the manufacturer's instructions.

Cosmid DNA isolation

Selected (Table 2) cosmids from the S. coelicolor A3(2) cosmid library (Redenbach et al., 1996) were obtained from Helen Kieser (John Innes Centre, Norwich, UK) as E. coli Sure cultures. Cosmid DNA was isolated from E. coli Sure using Wizard SV minipreps (Promega) according to the manufacturer's instructions. Cultures were grown at 37° C. in L broth containing ampicillin (50 μg/ml) and kanamycin (25 μg/ml) and isolated DNA transformed into E. coli JM109 by electroporation using a Biorad Gene Pulser according to the manufacturer's instructions on L agar plates containing ampicillin (50 μg/ml) and kanamycin (25 μg/ml). Cosmid DNA was again isolated using Wizard SV kits according to the manufactures instructions except that cultures were incubated in 2×YT broth containing ampicillin (50 μg/ml) and kanamycin (25 μg/ml) for exactly 18 hours at 250 rpm. DNA was eluted from the spin column twice with 40 μl of 10 mM Tris-HCL, pH 8.5 preheated to 50° C., quantified spectrophotometrically (OD₂₆₀) using a Beckman DU 650 spectrophotometer and stored at −70° C.

Purification of Tn5062 DNA

pQM5062 DNA was isolated using the Wizard SV minipreps (Promega) according to the manufacturer's instructions after growth in L broth containing ampicillin (50 μg/ml) and apramycin (100 μg/ml). Tn5062 was liberated from the plasmid by digestion with PvuII and electrophoresis on a 1% agarose gel made with TAE buffer. Following electrophoresis the 3442 bp Tn5062 band was excised from the gel using a scalpel and purified using the QIAEX II gel extraction system (Qiagen) according to manufacturer's instructions. Following purification, Tn5062 was further purified using the QIAquick PCR purification kit (Qiagen) and ethanol precipitated before being resuspended in 10 μl of sterile distilled water and quantified by comparison with known standards after agarose gel electrophoresis. Finally DNA was stored at −70° C.

Transposition Reaction

Transposition of Tn5062 into selected cosmids (Table 2) from the S. coelicolor cosmid library (Redenbach, et al., 1996) was carried out by preparing the following reaction mix, in the listed order, in an Eppendorf tube according to the manufacturer's instructions (Epicentre) and incubated for 2 hours at 37° C.:

| | |
|---|---|
| EZ::Tn 10x Reaction Buffer | 1 μl |
| S. coelicolor A3(2) cosmid DNA | 2.9 μl (200 ng or 7.6 × 10⁻⁹ μMoles) |
| Tn5062 DNA | 1.2 μl (17.5 ng or 7.6 × 10⁻⁹ μMoles) |
| Sterile distilled water | 3.9 μl |
| EZ::TN Transposase | 1 μl |

After completion, the reaction was stopped by adding 1 μl of EZ::TN stop solution and incubated at 70° C. for 10 minutes. 1 μl of the transposition reaction was then added to 40 μl of electrocompetent E. coli JM109 cells (prepared according to manufacturer's instructions) and electroporated using a Biorad Gene Pulser according to the manufacturer's instructions. Following electroporation cells were plated on L agar supplemented with ampicillin (50 μg/ml), kanamycin (25 μg/ml) and apramycin (100 μg/ml) to select for colonies containing transposed cosmids.

Isolation of transposed cosmid DNA

96 ampicillin, kanamycin and apramycin resistant colonies were picked and inoculated to a 96 square well growth block (ABgene), each well containing 1 ml of L broth containing ampicillin (50 μg/ml), kanamycin (25 μg/ml) and apramycin (100 μg/ml). The block was then incubated overnight at 37° C., 225 rpm. The next day 1.3 μl of each of the 96 cultures was the transferred to a second growth block, each well containing 1.3 ml of 2×YT supplemented with apramycin (1000 g/ml) and incubated for 18 hours at 37° C., 225 rpm. 330 μl of 60% (w/v) glycerol was then added to each of the 96 wells from the first growth block, mixed and stored at −70° C. Cosmid DNA was isolated from the cultures in the second growth block using the Wizard SV 96 kit from Promega and stored at −70° C.

Identification of transposition target sites by sequencing

Transposed cosmid DNA (1 μl) was first electrophoresed on a 0.7 (w/v) agarose gel to check DNA quality. For sequencing 10 μl from each of the 96 samples was transferred to a 96 well PCR plate (ABgene) and heated to 86° C. for 5 minutes in a MJ Research PTC-200 DNA engine. To each sample was then added 2 μl of transposon specific sequencing primer EZR1 (10 pmol/μl) (Table1, FIG. 1) and 8 μl of CEQ DTCS quick start sequencing kit (Beckman-Coulter). The sequencing reactions were then carried out in a MJ Research PTC-200 DNA engine by heating to 96° C. (20 seconds), 55° C. (20 seconds) and 60° C. (4 minutes) for 50 cycles. The samples were then analysed on a CEQ 200XL sequencer (Beckman-Coulter) using the long fast read program according to the manufacturer's conditions. Following sequencing the transposition target site was determined by comparison of each of the 96 sequences with the S. coelicolor A3(2) genome sequence at http://www.sanger.ac.uk/Projects/S coelicolor/(Bentley et al. 2002). Identified insertions in cosmid SC7C7 are shown in Table 3.

Transfer of insertion to S. coelicolor A3(2)

Replacement of a wild type gene with the cosmid-borne transposed copy was carried out by conjugation from E. coli according to Kieser et al. (2000). E. coli ET12567(pUZ8002) was grown in the presence of kanamycin (25 μg/ml) and chloramphenicol (25 μg/ml) and chemically competent cells prepared according to Sambrook et al. (1989). Selected transposed cosmids were then transformed into these cells (Sambrook et al., 1989) and grown on L agar supplemented with ampicillin (50 μg/ml), kanamycin (25 μg/ml) and apramycin (100 μg/ml) to select for colonies containing transposed cosmids. The next day a single transformant was inoculated into 10 ml of L broth containing apramycin (100 μg/ml), kanamycin (25 μg/ml) and chloramphenicol (25 μg/ml) and grown overnight at 37° C., 250 rpm. The next day 0.4 ml of the overnight culture was added to 39.6 ml of L broth supplemented with apramycin (100 μg/ml), kanamycin (25 μg/ml) and chloramphenicol (25 μg/ml), grown at 37° C. to an optical density of OD₆₀₀ 0.4-0.6. At this point, cells were harvested by centrifugation and washed twice in 40 ml of L broth, before being resuspended in 4 ml of L broth. Meanwhile, approximately 1×10⁸ of S. coelicolor M145 spores were added to 500 μl of 2×YT, heat shocked (50° C., 10 minutes) and allowed to cool. To this 500 μl of pregerminated spores was added 500 μl of the E. coli cells containing the transposed cosmid, after mixing, the cells and spores were centrifuged, most of the supernatant fraction removed and the pellet resuspended in the residual liquid. This was then plated on SFM agar, supplemented with 10 mM MgCl₂ and incubated at 30° C. for 16 hours. The next day the plate was overlayed with 1 ml of sterile distilled water supplemented with 1 mg of apramycin and 0.5 mg nalidixic acid and incubated at 30° C. for a further 3-4 days. After this time, individual transconjugants were picked off and patched onto SFM agar supplemented with nalidixic acid (25 μg/ml) and apramycin (100 μg/ml), similarly colonies were also patched onto SFM agar supplemented with nalidixic acid (25 μg/ml) and kanamycin (25 μg/ml). Those colonies that had undergone a gene replacement and replaced the wild type gene with the cosmid borne copy containing the insertion sequence were identified on the basis of apramycin resistance and kanamycin sensitivity.

Detection of eGFP expression in S. coelicolor A3(2)

Sterile coverslips were inserted into SFM agar at a 45° angle and 10 μl of S. coelicolor A3(2) spores were inoculated in the acute angle between coverslip and agar surface. After incubation for 1-7 days, coverslips were removed and washed twice by brief immersion in methanol. After drying, the coverslips were mounted on slides and examined microscopically using a Nikon Eclipse E600 fluorescence microscope. eGFP expression was observed by illumination with ultra violet light and fluorescence visualised with a FITC filter set.

Although the foregoing invention has been described in some detail by way of illustration and example for purposes of clarity and understanding, it will be readily apparent to those of ordinary skill in the art in light of the teachings of this invention that certain changes and modifications may be made thereto without departing from the spirit or scope of the appended claims.

For further information on techniques and materials, the addressee is referred also to general reference texts, such as Sambrook et al (1989), Kieser et al. (2000), Ausubel et al. (1989), and any later editions thereof (such as Sambrook and Russell (2001), as well as to the product literature of Epicentre and other suppliers of commercially available transposition systems.

Each publication and earlier application referred to herein is hereby incorporated by reference in its entirety and for all purposes.

TABLE 1
Oligonucleotides

| Ol- | |
|---|---|
| igo | Sequence |
| VC1 | GATCTGAATTCGGATCCTAATTAATTAATCTAGAAAGGAGGTGATCA |
| VC2 | TATGATCACCTCCTTTCTAGATTAATTAATTAGGATCCGAATTCA |
| VC3 | TATGGACGGAGCTCGGCCGCTTAAGGTACCGAATTCC |
| VC4 | TCGAGGAATTCGGTACCTTAAGCGGCCGAGCTCCGTCCA |
| EZ- | ATGCGCTCCATCAAGAAGAG |
| R1 | |

TABLE 2
Plasmids and cosmids

| Plasmid | Source |
|---|---|
| pET26B+ | Novagen |
| pALTER1 | Promega |
| pEGFP-N1 | Clontech |
| pHP45Ωaac | Blondelet-Rouault et al. |
| | (1997) |
| pMOD<MCS> | Epicentre |
| pIJ8660 | Sun et al. (1999) |
| pVC101 | This work |
| pVC102 | This work |
| pVC107 | This work |
| pQM501 | This work |
| pQM504 | This work |
| pQM5052 | This work |
| pQM5062 | This work |
| pUZ8002 | Kieser et al. (2000) |
| SC1A6 | Redenbach et al. (1996) |
| SC3A3 | Redenbach et al. (1996) |
| SC6A9 | Redenbach et al. (1996) |
| SC7B7 | Redenbach et al. (1996) |
| SC7C7 | Redenbach et al. (1996) |
| SCE59 | Redenbach et al. (1996) |
| SCF91 | Redenbach et al. (1996) |
| SCH63 | Redenbach et al. (1996) |
| SCH69 | Redenbach et al. (1996) |
| SCI7 | Redenbach et al. (1996) |
| SC4G10 | Redenbach et al. (1996) |
| SC4B10 | Redenbach et al. (1996) |
| SCH66 | Redenbach et al. (1996) |
| SC2E9 | Redenbach et al. (1996) |
| SC9B5 | Redenbach et al. (1996) |
| SCI51 | Redenbach et al. (1996) |
| 2SCI34 | Redenbach et al. (1996) |
| 2SCG38 | Redenbach et al. (1996) |
| SCC88 | Redenbach et al. (1996) |
| SCC77 | Redenbach et al. (1996) |
| SC9E12 | Redenbach et al. (1996) |
| SCF43 | Redenbach et al. (1996) |
| SC5C11 | Redenbach et al. (1996) |
| 2SCK8 | Redenbach et al. (1996) |
| SCH44 | Redenbach et al. (1996) |
| SC10F4 | Redenbach et al. (1996) |
| SCD66 | Redenbach et al. (1996) |
| SCE20 | Redenbach et al. (1996) |
| SCD16A | Redenbach et al. (1996) |
| SCH22A | Redenbach et al. (1996) |
| SCF55 | Redenbach et al. (1996) |
| SC3C3 | Redenbach et al. (1996) |
| SC2A11 | Redenbach et al. (1996) |

TABLE 3
Examples of transcriptional Tn5062 insertions in cosmid SC7C7

| | | | | | | Poten- | |
|---|---|---|---|---|---|---|---|
| | | | | | | tial | |
| | | | | | | eGFP | |
| | | | | | Inser- | Tran- | |
| | | Insertion | Insertion | | tion | scrip- | |
| | Trans- | Position | Position | | Strand | tion | Target |
| Cosmid | posant | (Genome) | (Cosmid) | Inserted ORF | (+/−) | (y/n/) | Site |
| SC7C7 | C12 | 6270990 | 295 | 13566 rRNA 6269992 . . . 6271519 + rrnE 16S | + | y | CCCTTGTGG |
| SC7C7 | F04 | 271341 | 646 | 13566 rRNA 6269992 . . . 6271519 + rrnE 16S | + | y | GTGAATAC |
| SC7C7 | A10 | 6271446 | 751 | 13566 rRNA 6269992 . . . 6271519 + rrnE 16S | − | n | CCTTCGAC |
| SC7C7 | G07 | 6272082 | 1387 | 13568 rRNA 6271800 . . . 6274919 + rrnE 23S | − | n | GTATACGG |
| SC7C7 | B10 | 272989 | 2294 | 13568 rRNA 6271800 . . . 6274919 + rrnE 23S | + | y | GTGCGTAAT |
| SC7C7 | H10 | 273051 | 2356 | 13568 rRNA 6271800 . . . 6274919 + rrnE 23S | + | y | GCCGAAGT |
| SC7C7 | C11 | 273249 | 2554 | 13568 rRNA 6271800 . . . 6274919 + rrnE 23S | + | y | GGTAAGTC |
| SC7C7 | G05 | 6273281 | 2586 | 13568 rRNA 6271800 . . . 6274919 + rrnE 23S | − | n | CCTGTCGGC |
| SC7C7 | E09 | 6273329 | 2634 | 13568 rRNA 6271800 . . . 6274919 + rrnE 23S | + | y | TCAAACAT |
| SC7C7 | A01 | 6273776 | 3081 | 13568 rRNA 6271800 . . . 6274919 + rrnE 23S | − | n | CGCTGGTC |
| SC7C7 | E12 | 6273823 | 3128 | 13568 rRNA 6271800 . . . 6274919 + rrnE 23S | − | n | CCTTACGG |
| SC7C7 | G03 | 6274456 | 3761 | 13568 rRNA 6271800 . . . 6274919 + rrnE 23S | − | n | CCTTTTATC |
| SC7C7 | E01 | 6274937 | 4242 | | − | ? | CAGTGGACG |
| SC7C7 | A04 | 6274964 | 4269 | | − | ? | GGTTGTTC |
| SC7C7 | E04 | 6275179 | 4484 | | + | ? | TTCCGTCAC |
| SC7C7 | E05 | 6275630 | 4935 | 13571 SCO5746, SC7C7.01 | − | n | ACTGCTGAT |
| | | | | 6275223 . . . 6275900 + hypothetical protein | | | |
| | | | | SC7C7.01 | | | |
| SC7C7 | E02 | 6275925 | 5230 | | + | ? | GGCTTGTTC |
| SC7C7 | D11 | 6277043 | 6348 | 13573 SCO5747, SC7C7.02c | − | y | GGCCCGACC |
| | | | | 6276106 . . . 6278856 − putative regulatory | | | |
| | | | | protein | | | |
| SC7C7 | C05 | 6277305 | 6610 | 13573 SCO5747, SC7C7.02c | + | n | GGTCGGGAC |
| | | | | 6276106 . . . 6278856 − putative regulatory | | | |
| | | | | protein | | | |
| SC7C7 | E08 | 6277864 | 7169 | 13573 SCO5747, SC7C7.02c | + | n | CCGATGAAC |
| | | | | 6276106 . . . 6278856 − putative regulatory | | | |
| | | | | protein | | | |
| SC7C7 | A05 | 6277949 | 7254 | 13573 SCO5747, SC7C7.02c | − | y | GTGCTGCAG |
| | | | | 6276106 . . . 6278856 − putative regulatory | | | |
| | | | | protein | | | |
| SC7C7 | F03 | 6277949 | 7254 | 13573 SCO5747, SC7C7.02c | + | n | GCGTAGACC |
| | | | | 6276106 . . . 6278856 − putative regulatory | | | |
| | | | | protein | | | |
| SC7C7 | G06 | 6278026 | 13573 | SCO5747,SC7C7.02c | + | n | GCGTAGACC |
| | | | | 6276106 . . . 6278856 − putative regulatory | | | |
| | | | | protein | | | |
| SC7C7 | A11 | 6278408 | 7713 | 13573 SC05747, SC7C7.02c | + | n | GCCGACCGC |
| | | | | 6276106 . . . 6278856 − putative regulatory | | | |
| | | | | protein | | | |
| SC7C7 | H03 | 6278566 | 13573 | SCO5747,SC7C7.02c | + | n | GCGTGGACC |
| | | | | 6276106 . . . 6278856 − putative regulatory | | | |
| | | | | protein | | | |
| SC7C7 | D06 | 6278734 | 8039 | 13573 SCO5747, SC7C7.02c | − | y | GTTCTGTGA |
| | | | | 6276106 . . . 6278856 − putative regulatory | | | |
| | | | | protein | | | |
| SC7C7 | D10 | 6279071 | 8376 | | + | ? | GATGAAGGT |
| SC7C7 | B04 | 6281301 | 10606 | 13576 SCO5748, SC7C7.03 | − | n | GCCACACAC |
| | | | | 6279265 . . . 6284754 + putative sensory | | | |
| | | | | histidine kinase | | | |
| SC7C7 | C04 | 6281495 | 10800 | 13576 SCO5748,SC7C7.03 | + | y | GGTCACGCG |
| | | | | 6279265 . . . 6284754 − putative sensory | | | |
| | | | | histidine kinase | | | |
| SC7C7 | F02 | 6281668 | 10973 | 13576 SCO5748, SC7C7.03 | − | n | CCCTTGGCG |
| | | | | 6279265 . . . 6284754 + putative sensory | | | |
| | | | | histidine kinase | | | |
| SC7C7 | H02 | 6281898 | 11203 | 13576 SCO5748,SC7C7.03 | + | y | GTCCAGGTG |
| | | | | 6279265 . . . 6284754 + putative sensory | | | |
| | | | | histidine kinase | | | |
| SC7C7 | A09 | 6282182 | 11487 | 13576 SCO5748, SC7C7.03 | − | n | CACCTGACC |
| | | | | 6279265 . . . 6284754 + putative sensory | | | |
| | | | | histidine kinase | | | |
| SC7C7 | G02 | 6282297 | 11602 | 13576 SCO5748, SC7C7.03 | + | y | GACCAGCTC |
| | | | | 6279265 . . . 6284754 + putative sensory | | | |
| | | | | histidine kinase | | | |
| SC7C7 | A07 | 6283214 | 12519 | 13576 SCO5748, SC7C7.03 | + | y | CCAGTCGTC |
| | | | | 6279265 . . . 6284754 + putative sensory | | | |
| | | | | histidine kinase | | | |
| SC7C7 | H11 | 6283268 | 12573 | 13576 SCO5748, SC7C7.03 | − | n | GTTCTGCTG |
| | | | | 6279265 . . . 6284754 + putative sensory | | | |
| | | | | histidine kinase | | | |
| SC7C7 | B06 | 6283391 | 12696 | 13576 SCO5748, SC7CT.03 | − | n | GTTCTGCTG |
| | | | | 6279265 . . . 6284754 + putative sensory | | | |
| | | | | histidine kinase | | | |
| SC7C7 | C01 | 6284449 | 13754 | 13576 SCO5748, SC7C7.03 | + | y | AGCACGGAC |
| | | | | 6279265 . . . 6284754 + putative sensory | | | |
| | | | | histidine kinase | | | |
| SC7C7 | D07 | 6284914 | 14219 | | + | ? | ACGTACGGG |
| SC7C7 | H09 | 6286360 | 15665 | 13583 SCO5750, SC7C7.05 | + | y | GTCTTCCGC |
| | | | | 6286097 . . . 6288886 + ftsK homolog | | | |
| SC7C7 | B03 | 6286832 | 16137 | 13583 SCO5750, SC7C7.05 | + | y | CGGCCACCC |
| | | | | 6286097 . . . 6288886 + ftsK homolog | | | |
| SC7C7 | F05 | 6287186 | 16491 | 13583 SCO5750, SC7C7.05 | + | y | TCGCCGACC |
| | | | | 6286097 . . . 6288886 + ftsK homolog | | | |
| SC7C7 | D03 | 6287254 | 16559 | 13583 SCO5750, SC7C7.05 | − | n | GCACCGGCG |
| | | | | 6286097 . . . 6288886 + ftsK homolog | | | |
| SC7C7 | A08 | 6288131 | 17436 | 13583 SCO5750, SC7C7.05 | + | y | ACTTCAACC |
| | | | | 6286097 . . . 6288886 + ftsK homolog | | | |
| SC7C7 | F11 | 6288234 | 17539 | 13583 SCO5750, SC7C7.05 | − | n | GGCCAGCTC |
| | | | | 6286097 . . . 6288886 + ftsK homolog | | | |
| SC7C7 | F09 | 6288477 | 17782 | 13583 SCO5750, SC7C7.05 | + | y | CTTCCTGCC |
| | | | | 6286097 . . . 6288886 + ftsK homolog | | | |
| SC7C7 | B02 | 6289171 | 18476 | | + | ? | CGCTCGAAA |
| SC7C7 | D08 | 6289754 | 19059 | 13587 SCO5751, SC7C7.06 | − | n | GGCTTGGGG |
| | | | | 6289190 . . . 6290053 + putative membrane | | | |
| | | | | protein | | | |
| SC7C7 | H04 | 6290058 | 19363 | | + | ? | GCGGGGACC |
| SC7C7 | C03 | 6290188 | 19493 | 13588 SCO5752, SC7C7.07 | − | n | GGCGCAGCC |
| | | | | 6290145 . . . 6291626 + conserved hypothetical | | | |
| | | | | protein SC7C7.07 | | | |
| SC7C7 | F07 | 6291027 | 20332 | 13588 SCO5752, SC7C7.07 | + | y | ACTTCGACC |
| | | | | 6290145 . . . 6291626 + conserved hypothetical | | | |
| | | | | protein SC7C7.07 | | | |
| SC7C7 | E11 | 6291102 | 20407 | 13588 SCO5752, SC7C7.07 | + | y | TCCTGGAGC |
| | | | | 6290145 . . . 6291626 + conserved hypothetical | | | |
| | | | | protein SC7C7.07 | | | |
| SC7C7 | E03 | 6291533 | 20838 | 13588 SCO5752, SC7C7.07 | − | n | CCATACGAC |
| | | | | 6290145 . . . 6291626 + conserved hypothetical | | | |
| | | | | protein SC7C7.07 | | | |
| SC7C7 | B01 | 6292335 | 21640 | 13591 SCO5753, pgsA 6291623 . . . 6292414 + | − | n | GTCCAGGCC |
| | | | | phosphatidylglycerophosphate synthase | | | |
| SC7C7 | B11 | 6293516 | 22821 | | + | ? | GTTTTCGCA |
| SC7C7 | A06 | 6293979 | 23284 | 13598 SCO5756, SC7C7.11 | − | n | GTCCACGAC |
| | | | | 6293651 . . . 6294121 + hypothetical protein | | | |
| | | | | SC7C7.11 | | | |
| SC7C7 | E10 | 6294345 | 23650 | 13601 SCO5757, SC7C7.12 | − | n | GCGCAGGGC |
| | | | | 6294128 . . . 6294394 + hypothetical protein | | | |
| | | | | SC7C7.12 | | | |
| SC7C7 | C07 | 6294633 | 23938 | 13602 SCO5758, SC7C7.13 | + | y | TGGTCAAGG |
| | | | | 6294450 . . . 6294824 + putative | | | |
| | | | | transcriptional regulator | | | |
| SC7C7 | D09 | 6295168 | 24473 | 13604 SCO5759, SC7C7.14 | − | n | CGGTGAGCG |
| | | | | 6295168 . . . 6295306 + hypothetical protein | | | |
| | | | | SC7C7.14 | | | |
| SC7C7 | C09 | 6295384 | 24689 | 13605 SCO5760, SC7C7.15c | + | n | GTGCGGGCC |
| | | | | 6295344 . . . 6296174 − DNA glycosylase | | | |
| SC7C7 | C08 | 6295844 | 25149 | 13605 SCO5760, SC7C7.15c | + | n | GTCCAGCAC |
| | | | | 6295344 . . . 6296174 − DNA glycosylase | | | |
| SC7C7 | B05 | 6295934 | 25239 | 13605 SCO5760, SC7C7.15c | − | y | GCGCTGGAG |
| | | | | 6295344 . . . 6296174 − DNA glycosylase | | | |
| SC7C7 | D02 | 6295943 | 25248 | 13605 SCO5760, SC7C7.15c | + | n | CGCGAACAC |
| | | | | 6295344 . . . 6296174 − DNA glycosylase | | | |
| SC7C7 | H06 | 6296183 | 25488 | | − | ? | CCCTTGAGT |
| SC7C7 | G04 | 6296468 | 25773 | 13606 SCO5761, SC7C7.16c | + | n | GGAGCCCGC |
| | | | | 6296193 . . . 6301265 − putative ATP − | | | |
| | | | | dependent DNA helicase | | | |
| SC7C7 | B08 | 6296627 | 25932 | 13606 SCO5761, SC7C7.16c | − | y | GTACGACAC |
| | | | | 6296193 . . . 6301265 − putative ATP − | | | |
| | | | | dependent DNA helicase | | | |
| SC7C7 | A03 | 6297137 | 26442 | 13606 SCO5761, SC7C7.16c | − | y | CGAGGAGAG |
| | | | | 6296193 . . . 6301265 − putative ATP − | | | |
| | | | | dependentDNA helicase | | | |
| SC7C7 | C02 | 6298130 | 27435 | 13606 SCO5761, SC7C7.16c | + | n | CGTGAAGGG |
| | | | | 6296193 . . . 6301265 − putative ATP − | | | |
| | | | | dependent DNA helicase | | | |
| SC7C7 | C06 | 6300217 | 29522 | 13606 SCO5761, SC7C7.16c | − | n | GAGCAGGC |
| | | | | 6296193 . . . 6301265 − putative ATP − | | | |
| | | | | dependent DNA helicase | | | |
| SC7C7 | F01 | 6301309 | 30614 | | − | ? | GCCACGCCC |
| SC7C7 | G09 | 6302647 | 417 | 13616 SCO5763, SC4H8.02 | − | n | CGGGAGGGC |
| | | | | 6302319 . . . 6303089 + putative membrane | | | |
| | | | | protein SCH4H8.02 | | | |
| SC7C7 | B07 | 6303689 | 1459 | 13619 SCO5765, SC4H8.04c | + | n | AGCACGGCG |
| | | | | 6303403 . . . 6304429 − hypothetical protein | | | |
| | | | | SCH4H8.04c | | | |
| SC7C7 | G01 | 6304465 | 2235 | 13621 SCO5766, SC4H8.05 | − | n | CCGTCAACC |
| | | | | 6304454 . . . 630468 + hypothetical protein | | | |
| | | | | SCH4H8.05 | | | |
| SC7C7 | G12 | 6304465 | 2235 | 13621 SCO5766, SC4H8.05 | − | n | CCGTCAACC |
| | | | | 6304454 . . . 630468 + hypothetical protein | | | |
| | | | | SCH4H8.05 | | | |

### REFERENCES

Ausubel F M, Brent R, Kingston R E, Moore D D, Seidman J G, Smith J A, Struhl K (1989) Current Protocols in Molecular Biology. Greene Publishing Associates and Wiley-Interscience, John Wiley & Sons, NY.

Bennett P M, Grinsted J, Richmond M H (1977) Mol.Gen.Genet., 154, 205-211.

Bentley, S. D., et al. (2002) Complete genome sequence of the model actinomycete Streptomyces coelicolor A3(2). Nature, 417, 141-147.

Berg D E, Howe M N (1989) (eds) “Mobile DNA” American Society for Microbiology, Washington, D.C., pages 163-184.

Blondelet-Rouault, M. H., Weiser, J., Lebrihi, A., Branny, P. and Pernodet, J. L. (1997). Antibiotic resistance cassettes derived from the omega interposon for use in E. coli and Streptomyces. Gene, 190, 315-317.

Davies D R et al. (2000) Science 289 (5476), 77.

Flett F, Mersinias V, Smith C P (1997) FEMS Microbiol. Lett., 155, 223-229.

Goryshin I Y and Reznikoff W S (1998) J Biol Chem 273, 7367.

Grindley N D, Read R R (1985) Annual Review of Biochemistry, 54, 863-896.

Kieser, T., Bibb, M. J., Buttner, M. J., Chater, K. F. and Hopwood, D. A. (2000). Practical Streptomyces Genetics, The John Innes Foundation, Norwich.

MacNeil, D. J., Gewain, K. M., Ruby, C. L., Dezeny, G., Gibbons, P. H. and MacNeil, T. (1992). Analysis of Streptomyces avermitilis genes required for avermectin biosynthesis utilizing a novel integration vector. Gene, 111, 61-68.

Matsushima ,P., Broughton, M. C., Turner, J. R. and Baltz, R. H. (1994) Gene, 146, 39-45.

Pansegrau W, Lanka E, Barth P T, Figurski D H, Guiney D G, Haas D, Helinski D R, Stanisich V A, Thomas C M (1994) J.Mol.Biol., 239, 623-663.

Redenbach, M., Kieser H. M., Denapaite, D., Eichner, A., Cullum, J., Kinashi, H. and Hopwood, D. A. (1996). A set of ordered cosmids and a detailed genetic and physical map for the 8 Mb Streptomyces coelicolor A3(2) chromosome. Molecular Microbiology, 21, 77-96.

Sambrook, Z., Fritsch, E. F., and Maniatis, T. (1989, 2001). Molecular Cloning: A Laboratory Manual, Cold Spring Harbor, N.Y.

Sambrook, Z., and Russell (2001). Molecular Cloning: A Laboratory Manual, Cold Spring Harbor, N.Y.

Sun, J., Kelleman G. H., Fernandez-Abalos J. M. and Bibb, M. J. (1999). Green fluorescent protein as a reprter for spatial and temporal expression in Streptomyces coelicolor A3(2). Microbiology, 145, 2221-2227.

Yanisch-Perron, C., Viera, J. and Messing J. (1985). Gene, 33,103-119.

We claim:

1. A nucleic acid construct comprising inverted repeat sequences of a transposable element and an origin of transfer, wherein the origin of transfer lies between the inverted repeat sequences, such that a transposition event involving the inverted repeat sequences will result in the origin of transfer being included in the resultant insertion at the transposition target site.

2. A construct according to claim 1, wherein the inverted repeat sequences are of, or derived from, the inverted repeat sequences of, a transposable element that employs a non-replicative transposition mechanism.

3. A construct according to claim 1, wherein the inverted repeat sequences are, or are derived from, the OE and/or IE inverted repeat sequences of the transposon Tn5.

4. A construct according to claim 3, wherein the inverted repeat sequences have at least 70% identity with any one or more of the following sequences:

| | |
|---|---|
| a) | 5′-CTGTC TCTTA TACAC ATCT-3′ |
| | 3′-GACAG AGAAT ATGTG TAGA-5′ |
| b) | 5′-CTGAC TCTTA TACAC AAGT-3′ |
| | 3′-GACTG AGAAT ATGTG TTCA-5′ |
| c) | 5′-CTGTC TCTTG ATCAG ATCTT GATC-3′ |
| | 3′-GACAG AGAAC TAGTC TAGAA CTAG-5′ |

5. A construct according to claim 4, wherein the inverted repeat sequences have at least 85% identity any one or more of said sequences a), b) and c).

6. A construct according to claim 3, wherein the inverted repeat sequences have the sequence:
5′-CTGTC TCTTA TACAC ATCT-3′
3′-GACAG AGAAT ATGTG TAGA-5′

7. A construct according to claim 1, which does not encode a transposase.

8. A construct according to claim 1, wherein the origin of transfer is an oriT which can be mobilised by the helper plasmids pUZ8002 and pUB307.

9. A construct according to claim 8, wherein the origin of transfer has the sequence:
CCGGGCAGGA TAGGTGAAGT AGGCCCACCC GCGAGCGGGT
GTTCCTTCTT CACTGTCCCT TATTCGCACC TGGCGGTGCT
CAACGGGAAT CCTGCTCTGC GAGGCTGGC,
or a variant thereof having origin of transfer function.

10. A construct according to claim 1, which comprises a promoterless reporter gene located between the inverted repeat sequences.

11. A construct according to claim 10, wherein the promoterless reporter gene is operatively associated with a ribosome binding site and the construct further comprises, upstream of the reporter gene and ribosome binding site and between the inverted repeat sequences, a translational stop sequence.

12. A construct according to claim 1, which lacks an origin of replication.

13. A construct according to claim 1, which is linear and consists essentially of the inverted repeat sequences and any sequences located therebetween.

14. A vector including a construct according to claim 1.

15. A vector according to claim 14, which includes PCR primer binding sites and/or restriction sites for the amplification or excision from the vector of a linear nucleic acid consisting essentially of the construct.

16. A method for mutagenising DNA of interest from a bacterial species, the method comprising:
a) contacting said DNA of interest with a nucleic acid construct according to claim 1, to form a transposition mixture;
b) incubating the transposition mixture under conditions suitable for transposition to occur, said contacting and incubating steps being performed other than within cells of said bacterial species;
c) transferring transposed DNA of said transposition mixture by conjugation from a donor bacterial cell into a host bacterial cell; and
d) incubating the host cell under conditions suitable for homologous recombination between the transposed DNA and the DNA of the host cell.

17. A method according to claim 16, wherein the transposition mixture also includes a transposase.

18. A method according to claim 17, wherein the transposase is, or is derived from, Tn5 transposase.

19. A method according to claim 18, wherein the transposase is hyperactive mutant Tn5 transposase.

20. A method according to claim 16, wherein the DNA of interest is contained in one or more circular DNA molecules.

21. A method according to claim 16, wherein the construct is linear.

22. A method according to claim 16, wherein the contacting and incubating steps (a) and (b) occur outside any bacterial cell, and the method comprises the further step (b1) of transferring the transposed DNA of the transposition mixture into the bacterial donor cell, prior to the conjugation step (c) into the host cell.

23. A method according to claim 16, which comprises an additional step, before the conjugation step, of identifying the site in the DNA of interest at which a transposition event has led to an insertion.

24. A method according to claim 16, wherein the DNA of interest is DNA from a bacterial library.

25. A method according to claim 24, wherein the DNA from which the library is generated is from bacteria of the genus Streptomyces.

26. A method according to claim 16, wherein the host cell is a pre-germinated spore.

27. A method according to claim 16, wherein the host cell is of the species or strain from which the DNA of interest originates.

28. A method according to claim 16, wherein the donor cell is of a different cell type from the host cell.

29. A method according to claim 16, which comprises an additional step (e) of detecting whether homologous recombination has occurred in the host cell.

30. A method according to claim 29, wherein the additional step (e) comprises: detecting the loss in the host cell of a selectable marker that is borne by the DNA of interest; and detecting the retention of a selectable marker that is borne by the construct.

31. A method according to claim 16, wherein the method comprises, prior to the conjugation step, an additional step of replacing part or all of the transposition-derived insert by a further step of homologous recombination, to remove sequences from the insert and/or to add sequences to the insert.

32. A method according to claim 16, which is carried out simultaneously on several DNA molecules of interest, which are conjugated from different donor cells into different host cells, to produce a plurality of independently mutated host cells.

33. A method for mutagenising DNA of interest of a bacterial species, the method comprising the steps of:
a) contacting said DNA of interest with a nucleic acid construct according to claim 1, to form a transposition mixture;
b) incubating the transposition mixture under conditions suitable for transposition to occur, said contacting and incubating steps being performed other than within cells of said bacterial species; and
c) storing transposed DNA of said transposition mixture for future use.

34. A method for mutagenising DNA of interest of a bacterial species, the method comprising, following the production and storage of transposed DNA according to claim 33 above, the steps of:
a) transferring said transposed DNA by conjugation from a donor bacterial cell into a host bacterial cell; and
b) incubating the host cell under conditions suitable for homologous recombination between the transposed DNA and the DNA of the host cell.

35. A host cell producible or as produced by the method of claim 16.

36. A method of determining the effect of a genetic disruption, the method comprising culturing a host cell producible or as produced by the method of claim 16, and determining the effect of the disruption on the cell.

37. Transposed DNA of interest producible or as produced by the process according to claim 33.

38. Transposed DNA according to claim 37 contained in a bacterial cell or cells.

39. A host cell producible or as produced by the method of claim 34.

40. A method of determining the effect of a genetic disruption, the method comprising culturing a host cell producible or as produced by the method of claim 34, and determining the effect of the disruption on the cell.


SEQUENCE LISTING (13 sequences)

SEQ ID NO: 1 - DNA, 19 bp, Artificial Sequence
  Synthetic sequence
ctgtctcttatacacatct

SEQ ID NO: 2 - DNA, 19 bp, Artificial Sequence
  Synthetic sequence
agatgtgtataagagacag

SEQ ID NO: 3 - DNA, 19 bp, Artificial Sequence
  Synthetic sequence
ctgactcttatacacaagt

SEQ ID NO: 4 - DNA, 24 bp, Artificial Sequence
  Synthetic sequence
ctgtctcttgatcagatcttgatc

SEQ ID NO: 5 - DNA, 109 bp, Artificial Sequence
  Synthetic sequence
ccgggcaggataggtgaagtaggcccacccgcgagcgggtgttccttcttcactgtcccttattcgcacctggcggtgctcaacgggaatcctgctctgcgaggctggc

SEQ ID NO: 6 - DNA, 47 bp, Artificial Sequence
  Synthetic sequence
gatctgaattcggatcctaattaattaatctagaaaggaggtgatca

SEQ ID NO: 7 - DNA, 45 bp, Artificial Sequence
  Synthetic sequence
tatgatcacctcctttctagattaattaattaggatccgaattca

SEQ ID NO: 8 - DNA, 37 bp, Artificial Sequence
  Synthetic sequence
tatggacggagctcggccgcttaaggtaccgaattcc

SEQ ID NO: 9 - DNA, 39 bp, Artificial Sequence
  Synthetic sequence
tcgaggaattcggtaccttaagcggccgagctccgtcca

SEQ ID NO: 10 - DNA, 20 bp, Artificial Sequence
  Synthetic sequence
atgcgctccatcaagaagag

SEQ ID NO: 11 - DNA, 19 bp, Artificial Sequence
  Synthetic sequence
acttgtgtataagagtcag

SEQ ID NO: 12 - DNA, 24 bp, Artificial Sequence
  Synthetic sequence
gatcaagatctgatcaagagacag

SEQ ID NO: 13 - DNA, 3442 bp, Artificial Sequence
  Synthetic sequence
ctgtctcttatacacatctcaaccatcatcgatgaattcggatcctaattaattaatctagaaaggaggtgatcatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcttaaggtaccgaattcgagggggatccggtgattgattgagcaagctttatgcttgtaaaccgttttgtgaaaaaatttttaaaataaaaaaggggacctctagggtccccaattaattagtaatataatctattaaaggtcattcaaaaggtcatccaccggatcagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtgttgccccagcaatcagcgcgaccttgcccctccaacgtcatctcgttctccgctcatgagctcagccaatcgactggcgagcggcatcgcattcttcgcatcccgccctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagccggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataaagcttgctcaatcaatcaccggatccccgacctgcaggtcgacttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatccccggggacctgcaggcatgcaagcttcagggttgagatgtgtataagagacag